(19) United States
(12) Patent Application Publication
(10) Pub. No.: US 2024/0221001 A1
(43) Pub. Date: Jul. 4, 2024

(54) CARTRIDGE AUTHENTICATION METHOD

(71) Applicant: Shenzhen Lexon Electronic Technology Co.,Ltd, Shenzhen (CN)

(72) Inventor: ZHENGWU TAN, Shenzhen (CN)

(21) Appl. No.: 18/205,068

(22) Filed: Jun. 2, 2023

(30) Foreign Application Priority Data
Dec. 30, 2022 (CN) 202211744094.4

(51) Int. Cl. G06Q 30/018; A24F 40/53

(52) U.S. Cl. CPC G06Q 30/0185; A24F 40/53

(57) ABSTRACT

The present disclosure provides a cartridge authentication method, including a cigarette rod unit and a cartridge unit. The cigarette rod unit comprises an access detection circuit, a current-limiting protection circuit, a voltage-limiting protection circuit, a data transmitting circuit and a data receiving circuit. The cartridge unit includes an access detection circuit, a polarity protection circuit, a data transmitting circuit, a data receiving circuit, a heating circuit and an MCU. The present disclosure is simple in structure, with fewer components utilized and high degree of integration and low cost of manufacturing, which does not change the two-wire connection mode of traditional electronic cigarettes and can be widely promoted and applied.

### CROSS-REFERENCE TO RELATED APPLICATIONS

This application claims the benefit and priority of Chinese patent application No. 202211744094.4, filed on Dec. 30, 2022, disclosure of which is hereby incorporated by reference in its entirety.

### TECHNICAL FIELD

The present disclosure relates to the technical field of two-wire communications, more particularly to a cartridge authentication method.

### BACKGROUND

With the development of technologies, electronic cigarettes are increasingly used, which creates a huge electronic cigarette market. However, most of the cartridge products for electronic cigarettes available on the market have no anti-counterfeiting function. Even though anti-counterfeiting labels are employed for identification, the anti-counterfeiting labels may still be fake, resulting in a great number of cartridge counterfeits on the market, among which some inferior products are of great harm to human body.

Through retrieval it is found that the prior patent discloses an electronic cigarette cartridge authentication method and device and an electronic cigarette thereof. The electronic cigarette cartridge authentication method includes: acquiring a random number sent by an electronic cigarette cartridge; scrambling a current plaintext adopting the random number to obtain a first scrambled plaintext; performing encryption on the first scrambled plaintext using an RC4 algorithm to obtain first MAC data; and comparing the first MAC data with second MAC data sent by the electronic cigarette cartridge, and determining whether the electronic cigarette cartridge passes authentication or not. Due to the fact that the RC4 algorithm is adopted that is low in hardware resource consumption, the technical problem that the electronic cigarette cartridge authentication method is high in cost is effectively solved. In addition, the plaintext is scrambled by the random number, which effectively improves the authentication security. However, the internal structure is complex, with too many components utilized and high cost of manufacturing, which is not conducive to wide promotion and application.

Furthermore, in the traditional electronic cigarettes available on the market, a cigarette rod and a cartridge are basically connected through a two-wire connection mode. Due to the limitation by designs, it is needed to make a cigarette rod and a cartridge matched before normal usage, where the matching approaches all employ wireless modes such as NFC or change the two-wire connection into a four-wire or multiwire connection.

### SUMMARY

### (1) Technical Problems to be Solved

In view of the drawbacks of the existing technologies, the present disclosure provides a cartridge authentication method, which addresses the problems put forward in the background.

### (2) Technical Solution

In order to achieve the above aim, the present disclosure is implemented through the following technical solution. A cartridge authentication method includes a cigarette rod unit and a cartridge unit, wherein the cigarette rod unit includes an access detection circuit, a current-limiting protection circuit, a voltage-limiting protection circuit, a data transmitting circuit and a data receiving circuit; and the cartridge unit includes an access detection circuit, a polarity protection circuit, a data transmitting circuit, a data receiving circuit, a heating circuit and an MCU.

Preferably, the cigarette rod unit and the cartridge unit are connected through a two-core circuit, and the two-core circuit is divided into an authentication period and a power supply period in terms of transmission time.

Preferably, when the transmission time is at the power supply period, the cigarette rod unit takes responsibility for the power supply to the cartridge unit and a bus maintains a high level.

Preferably, the cigarette rod unit receives a communication signal by calculating a resistance value or a current of the bus.

Preferably, the cartridge transmits a communication signal by changing a resistance value or a current of the bus.

Preferably, based on a method for insertion detection and encryption docking between a cigarette rod and a cartridge in an electronic cigarette with time division multiplexing of power supply and communication, a cigarette rod authentication scheme includes:
a. when the cigarette rod is in a standby state and the cartridge is not inserted, inputting a high level by the access detection circuit;
b. when the cartridge is inserted, the level of the access detection circuit of the cigarette rod being lowered by the cartridge, completing cartridge insertion and starting an authentication phase; and
c. at the authentication phase, transmitting authentication data first by the cartridge that includes an authentication code, cartridge attributes and the like data; after receiving the data, parsing the data by the cigarette rod to separate out the authentication code as cartridge attribute data; if the authentication code is correct, saving the cartridge attribute data, completing authentication and starting a normal power supply smokable state; if the authentication code is incorrect, which is indicative of authentication failure, shutting down power supply and communication.

Preferably, based on a method for insertion detection and encryption docking between a cigarette rod and a cartridge in an electronic cigarette with time division multiplexing of power supply and communication, a cartridge authentication scheme includes:
a. when the cartridge is in a standby state, all components being not operating, where no power supply circuit is required to provide for the cartridge;
b. when the cartridge is inserted into the cigarette rod, providing a low level for the cigarette rod by the access detection circuit;
c. when the cartridge detects a power supply, starting to transmit an authentication code and cartridge attribute data; and
d. if the authentication code is correct, completing authentication and entering a normal heating smokable state; if the authentication code is incorrect, which is indicative of authentication failure, shutting down power supply and communication of the cartridge.

The two-core circuit is divided into an authentication period and a normal power supply period in terms of transmission time. When the bus is at the authentication period, the cigarette rod transmits an authentication communication signal by lowering the level of the bus, and the cartridge uploads an authentication communication signal by lowering the level of the bus. When the bus is at the normal power supply period, the bus takes responsibility for the slave power supply and maintains a high level. When the bus is at a signal transmission timeslot, the cigarette rod unit shuts down the bus power supply, the bus level is in a high level pull-up state. Both uplink and downlink of signals employ voltage signals, which eliminates the interference of a slave load change to a bus signal. Parameters such as the slave power supply capability, the bus voltage value, the communication distance, etc. only depend on the selection of device parameters. The limitation of the traditional two buses on the bus voltage and the slave power supply capability is broken. There is no need of dedicated modem chips and no need of changing traditional structures. The application cost and the technical threshold are greatly reduced, and the application scope is broadened.

### (3) Beneficial Effects

The present disclosure provides a cartridge authentication method, which has the following beneficial effects.

The present disclosure is simple in structure, with fewer components utilized and high degree of integration and low cost of manufacturing, which does not change the two-wire connection mode of traditional electronic cigarettes and can be widely promoted and applied.

### BRIEF DESCRIPTION OF DRAWINGS

FIG. 1 is a flowchart of operation of a cigarette rod of the present disclosure.

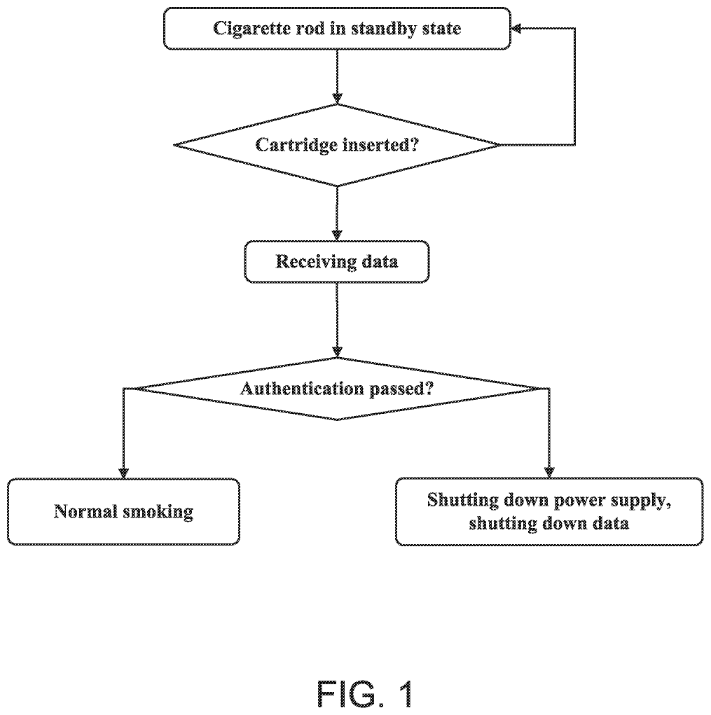

FIG. 2 is a flowchart of operation of a cartridge of the present disclosure.

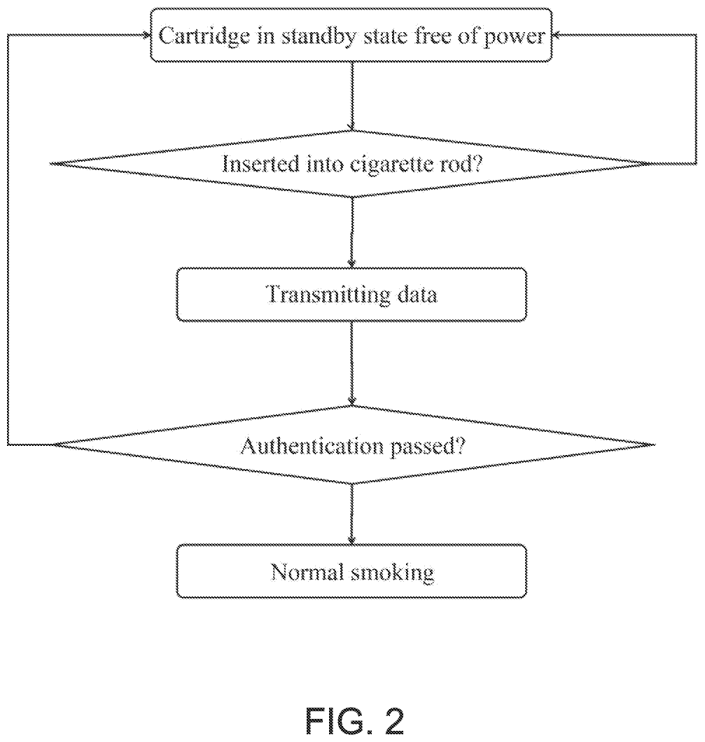

FIG. 3 is a circuit diagram of operation of a cigarette rod of the present disclosure.

FIG. 4 is a circuit diagram of operation of a cartridge of the present disclosure.

FIG. 5 is a circuit diagram of an interface of a cartridge unit of the present disclosure.

### DETAILED DESCRIPTION

The technical solutions of the embodiments of the present disclosure will be clearly and completely described below with reference to the drawings of the embodiments of the present disclosure. Apparently, the described embodiments are merely part embodiments of the present disclosure, rather than all of the embodiments. All of the other embodiments that a person skilled in the art obtains on the basis of the embodiments of the present disclosure without paying creative work fall within the protection scope of the present disclosure.

### Embodiment 1

A cartridge authentication method, including a cigarette rod unit and a cartridge unit, wherein the cigarette rod unit includes an access detection circuit, a current-limiting protection circuit, a voltage-limiting protection circuit, a data transmitting circuit and a data receiving circuit; and

the cartridge unit includes an access detection circuit, a polarity protection circuit, a data transmitting circuit, a data receiving circuit, a heating circuit and an MCU.

The cigarette rod unit and the cartridge unit are connected through a two-core circuit, and the two-core circuit is divided into an authentication period and a power supply period in terms of transmission time.

When the transmission time is at the power supply period, the cigarette rod unit takes responsibility for the power supply to the cartridge unit and a bus maintains a high level.

The cigarette rod unit receives a communication signal by calculating a resistance value or a current of the bus.

The cartridge transmits a communication signal by changing a resistance value or a current of the bus.

Based on a method for insertion detection and encryption docking between a cigarette rod and a cartridge in an electronic cigarette with time division multiplexing of power supply and communication, a cigarette rod authentication scheme includes:
a. when the cigarette rod is in a standby state and the cartridge is not inserted, the access detection circuit inputs a high level;
b. when the cartridge is inserted, the level of the access detection circuit of the cigarette rod is lowered by the cartridge, the cartridge insertion is completed, and an authentication phase is started; and
c. at the authentication phase, the cartridge first transmits authentication data that includes an authentication code, cartridge attributes and the like data; after receiving the data, the cigarette rod parses the data to separate out the authentication code as cartridge attribute data; if the authentication code is correct, the cartridge attribute data is saved, the authentication is completed, and a normal power supply smokable state is started; if the authentication code is incorrect, which is indicative of authentication failure, power supply and communication are shut down.

Based on a method for insertion detection and encryption docking between a cigarette rod and a cartridge in an electronic cigarette with time division multiplexing of power supply and communication, a cartridge authentication scheme includes:
a. when the cartridge is in a standby state, all components are not operating, where no power supply circuit is required to provide for the cartridge;
b. when the cartridge is inserted into the cigarette rod, the access detection circuit provides a low level for the cigarette rod;
c. when the cartridge detects a power supply, the cartridge starts to transmit an authentication code and cartridge attribute data; and
d. if the authentication code is correct, the authentication is completed, and a normal heating smokable state is started; if the authentication code is incorrect, which is indicative of authentication failure, power supply and communication are shut down for the cartridge.

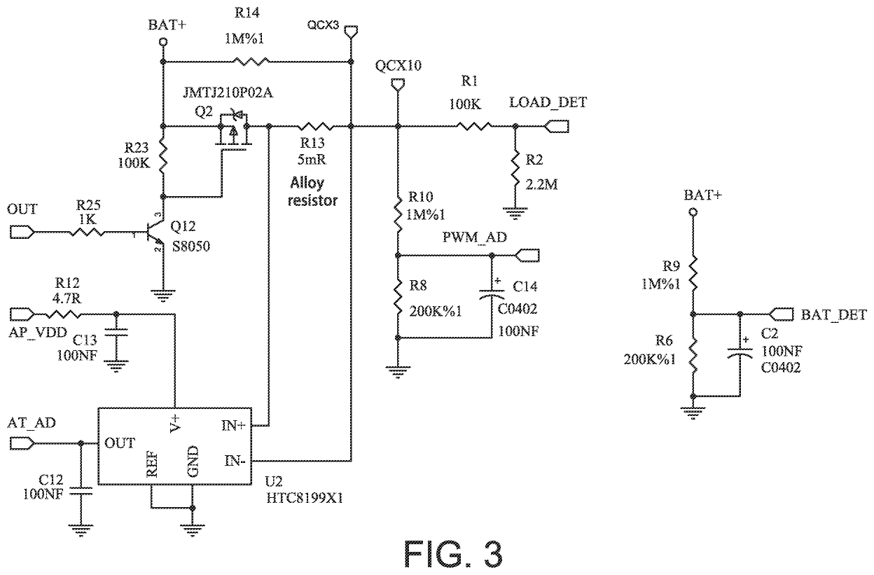

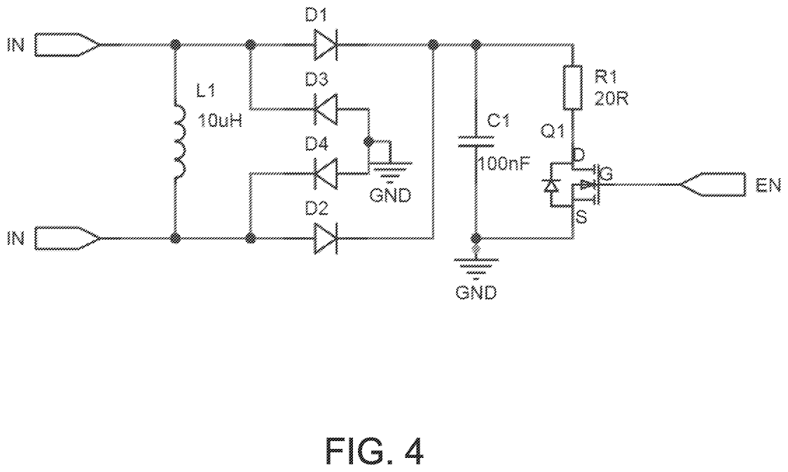

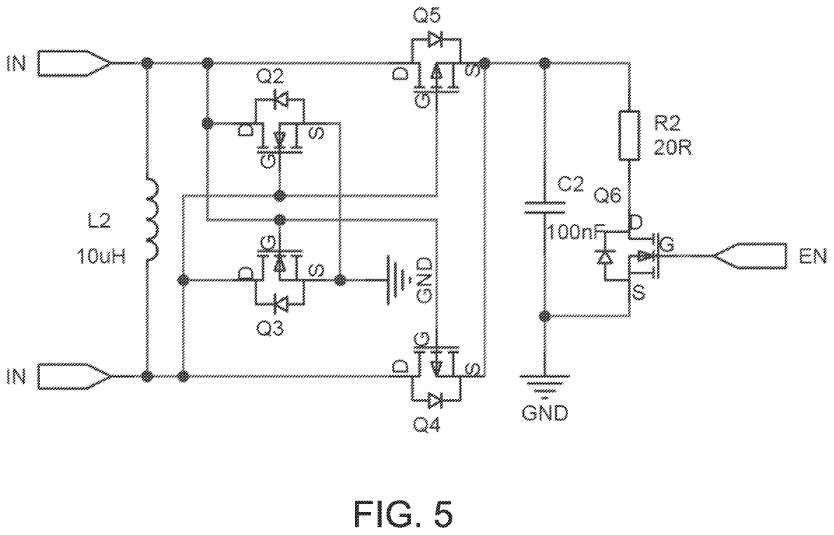

As shown in FIG. 3 and FIG. 4, the present embodiment provides a novel specific circuit structure based on the method for insertion detection and encryption docking between a cigarette rod and a cartridge in an electronic cigarette with time division multiplexing of power supply and communication.
1. FIG. 3 is a circuit diagram of an interface of a cigarette rod. The cigarette rod includes an access detection circuit, a current-limiting protection circuit, a voltage-limiting protection circuit and a data receiving circuit, wherein
a. R1, R2 and R4 in the figure form the access detection circuit, which provides a low level for an MCU when a cartridge is inserted;
b. R12, C13, U2, C12 and R13 in the figure form the current-limiting protection circuit, which provides current-limiting protection and the data receiving circuit at the system power supply phase; and
c. R10, R8 and C14 in the figure form the voltage-limiting protection circuit, which provides voltage-limiting protection at the system power supply phase.
2. FIG. 4 is a circuit diagram of an interface of a cartridge unit. The cartridge unit includes an access detection circuit, a polarity protection circuit and a data transmitting circuit, wherein
a. L1 in the figure forms the access detection circuit and a heating circuit;
b. D1, D2, D3 and D4 in the figure form the polarity protection circuit; and
c. R1 and Q1 in the figure form the data transmitting circuit.
3. FIG. 5 is a circuit diagram of an interface of a cartridge unit, which is a derived circuit diagram of the cartridge circuit shown in FIG. 4 to reduce line-drop, including an access detection circuit, a polarity protection circuit and a data transmitting circuit, wherein
a. L2 in the figure forms the access detection circuit and a heating circuit;
b. Q2, Q3, Q4 and Q5 in the figure form the polarity protection circuit; and
c. R2 and Q6 in the figure form the data transmitting circuit.

The present disclosure is simple in structure, with fewer components utilized and low cost of manufacturing, which can be widely promoted and applied.

The above shows and describes the basic principle, the main features and the advantages of the present disclosure. For those skilled in the art, apparently, the present disclosure is not limited to the details of the above exemplary embodiments, and the present disclosure may be implemented by other specific forms without departing from the spirit or basic features of the present disclosure. Therefore, no matter from which point, the embodiments should be viewed as exemplary, but restrictive. The scope of the present disclosure is limited by claims appended herein rather than by the description above. Therefore, all changes falling within the implication and scope of equivalent elements of the claims are intended to be included in the present disclosure. Any designator in the claims should not be viewed as a limitation to the claims.

In addition, it should be understood that although the present description is described according to embodiments, it is not that each embodiment contains only one independent technical scheme. The narration mode of the present description is merely for the purpose of clarity. Those skilled in the art should view the present description as an entirety. Technical schemes in each embodiment may be appropriately combined to form other embodiments that can be understood by those skilled in the art.

What is claimed is:

1. A cartridge authentication method, comprising a cigarette rod unit and a cartridge unit, wherein the cigarette rod unit comprises an access detection circuit, a current-limiting protection circuit, a voltage-limiting protection circuit, a data transmitting circuit and a data receiving circuit;
the cartridge unit comprises an access detection circuit, a polarity protection circuit, a data transmitting circuit, a data receiving circuit, a heating circuit and an MCU; and
the cigarette rod unit and the cartridge unit are connected through a two-core circuit, and the two-core circuit is divided into an authentication period and a power supply period in terms of transmission time; when the transmission time is at the power supply period, the cigarette rod unit takes responsibility for the power supply to the cartridge unit and a bus maintains a high level.

2. The cartridge authentication method according to claim 1, wherein the cigarette rod unit receives a communication signal by calculating a resistance value or a current of the bus; the cartridge transmits a communication signal by changing a resistance value or a current of the bus; and based on a method for insertion detection and encryption docking between a cigarette rod and a cartridge in an electronic cigarette with time division multiplexing of power supply and communication, a cigarette rod authentication scheme comprises:
a. when the cigarette rod is in a standby state and the cartridge is not inserted, inputting a high level by the access detection circuit;
b. when the cartridge is inserted, the level of the access detection circuit of the cigarette rod being lowered by the cartridge, completing cartridge insertion and starting an authentication phase; and
c. at the authentication phase, transmitting authentication data first by the cartridge that comprises an authentication code, cartridge attributes and the like data; after receiving the data, parsing the data by the cigarette rod to separate out the authentication code as cartridge attribute data; if the authentication code is correct, saving the cartridge attribute data, completing authentication and starting a normal power supply smokable state; if the authentication code is incorrect, which is indicative of authentication failure, shutting down power supply and communication.

3. The cartridge authentication method according to claim 1, wherein based on a method for insertion detection and encryption docking between a cigarette rod and a cartridge in an electronic cigarette with time division multiplexing of power supply and communication, a cartridge authentication scheme comprises:
a. when the cartridge is in a standby state, all components being not operating, where no power supply circuit is required to provide for the cartridge;
b. when the cartridge is inserted into the cigarette rod, providing a low level for the cigarette rod by the access detection circuit;
c. when the cartridge detects a power supply, starting to transmit an authentication code and cartridge attribute data; and
d. if the authentication code is correct, completing authentication and entering a normal heating smokable state; if the authentication code is incorrect, which is indicative of authentication failure, shutting down power supply and communication of the cartridge.
